(19) United States
(12) Patent Application Publication
(10) Pub. No.: US 2006/0173081 A1
(43) Pub. Date: Aug. 3, 2006

(54) Treating morning migraines with propranolol

(76) Inventors: Keith S. Rotenberg, Denville, NJ (US); George Bobotas, Tarpon Springs, FL (US)

Correspondence Address: ARENT FOX PLLC, 1050 CONNECTICUT AVENUE, N.W., SUITE 400, WASHINGTON, DC, 20036, US

(21) Appl. No.: 11/239,687

(22) Filed: Sep. 30, 2005

Related U.S. Application Data
(60) Provisional application No. 60/614,545, filed on Oct. 1, 2004.

(51) Int. Cl. A61K 31/138

(52) U.S. Cl. 514/651

(57) ABSTRACT

This invention relates to a method for preventing and treating morning migraine headaches. Pursuant to this method, a therapeutic amount of β-adrenergic-blocking agent is administered nightly to a person that suffers from migraine attacks such that the blocking agent is first released during morning hours when the person is most susceptible to morning migraine.

This nonprovisional application claims the benefit of U.S. Provisional Application No. 60/614,545, filed Oct. 1, 2004.

### BACKGROUND OF THE INVENTION

1. Field of Invention

The present invention relates to a novel method for prevention and treatment of morning migraines. A pharmaceutical composition containing a β-adrenergic-blocking agent, such as propranolol or a salt thereof, and one or more pharmaceutically acceptable excipients is administered in the evening to a person to prevent morning migraine or to maintain a morning migraine preventing amount in the person during the morning hours.

2. Description of the Related Art

Migraine is a common syndrome characterized by recurrent paroxysmal attacks of headache, often throbbing in character and sometimes, but not invariably, unilateral in distribution. The attacks often last for hours, less commonly for several days. This headache is severe and may be quite incapacitating. Its pain is frequency accompanied by photophobia, nausea, vomiting, and prostration.

More than 28 million Americans suffer from migraine headaches. Nearly 12 million of those Americans report that they have experienced “morning migraine,” a condition in which moderate to severe pain and other symptoms force patients to awaken from sleep.

β-adrenergic-blocking agents such as the propranolol product Inderal®, certain dihydropyridine compounds and divalproex sodium are known for the prophylaxis of migraine. However, these agents have not generally been shown to be effective in the management of the symptoms of an acute migraine attack. Once the attack has commenced, it has been widely reported that it is too late to administer these agents. In this circumstance, a treatment of choice becomes a drug such as an ergotamine.

As a result of the foregoing, a normal procedure for individuals subject to migraines involves the daily administration of a prophylactic dosage of a β-adrenergic-blocking agent, such as propranolol. This essentially involves maintaining a therapeutic level or concentration of blocking agent in a person's bloodstream on a long term basis which may be months in duration.

That procedure has been shown to be effective in reducing the frequency and severity of migraine attacks in humans. A disadvantage, however, is the requirement for virtually constant drug therapy.

Another disadvantage of that procedure involves individuals having certain medical complications. For example, those who are pregnant, suffering hepatic impairment or having bronchitis or emphysema can be subjected to its long term, virtually constant drug exposure only under closely monitored conditions, if at all. Consequently, many prospective patients are precluded from the benefits of that procedure.

### SUMMARY OF THE INVENTION

In view of the foregoing, it is apparent that a serious need exists for an improved method for utilizing β-adrenergic-blocking agents for the control of migraine. Thus, it is an object of the present invention to devise a method whereby the need for administration of the blocking agents can be targeted so that the agents are effective to prevent migraine during to a particular period in the day, namely the morning hours. A further object of this invention involves the reduction in total dosage amount, so as to minimize the adverse reactions to this drug therapy.

It has been discovered that the foregoing objectives may be achieved in the treatment of certain migraines through the selective and acute administration of β-adrenergic-blocking agents. More specifically, a therapeutically effective amount of a blocking agent is provided to a person to be effective during the morning hours, for example, between 12:00 midnight and 12:00 noon, preferably between 5:00 AM and 12:00 noon. In certain embodiments, the Cₘₐₓ is achieved between the hours of 8:00 am and 2:00 pm, preferably between 9:00 am and 12:00 noon.

It has also been discovered that the β-adrenergic-blocking agent is desirably provided using a delayed/extended release composition suitable for oral administration. This ensures that the β-adrenergic-blocking agent can be taken before a person goes to bed, and the active composition is not released to the sites governing migraine attack until a number of hours later when the person is more likely to be subjected to morning migraine.

It has further been discovered that, upon administration as aforesaid, β-adrenergic-blocking agents function in more than a prophylactic manner. In accordance with the method of the present invention, these blocking agents successfully prevent and treat morning migraine headaches.

Thus, the embodiments of the present invention include a method for treatment of a patient that suffers from morning migraines comprising administering a pharmaceutical composition containing a β-adrenergic-blocking agent and one or more pharmaceutically acceptable excipients to said patient in an amount sufficient to provide a therapeutically effective amount of said blocking agent in the morning to prevent or treat morning migraines. Preferred blocking agents may include propranolol, nadolol, timolol, matoprolol, atenolol, labetolol, pindolol, oxprenolol or a salt thereof. In a preferred embodiment, the blocking agent is propranolol or a salt thereof, more preferably propranolol hydrochloride.

In one embodiment, the pharmaceutical composition of the invention is administered in the evening, preferably between 6:00 PM and 12:00 midnight, more preferably between 8:00 PM and 12:00 midnight, and even more preferably at about 10:00 PM. In one embodiment, the composition of the invention is administered each night for an extended period of nights.

In one embodiment, administration commences in the evening and release of the propranolol or salt thereof is delayed until a period of time during morning hours of the day. In a preferred embodiment, the composition is administered in the evening before a person goes to bed, and blocking agent is first released, preferably from a pill or capsule, during the early morning hours, preferably between 12:00 midnight and 4:00 am.

In one embodiment, the administration provides an effective amount of blocking agent available at least for a time period between approximately 12:00 midnight to 12:00 noon, preferably between approximately 4:00 AM and 12:00 noon, more preferably between approximately 5:00 AM and 12:00 noon, and most preferably between approximately 6:00 AM to 11:00 AM.

In one embodiment, administration of the pharmaceutical composition of the invention occurs orally. The blocking agent is preferably administered in a nontoxic pharmaceutically acceptable dosage. In one embodiment, the blocking agent is administered in a pill or capsule. The pill or capsule may contain a delayed-release component and a controlled-release component. Preferably, the delayed-release component is a delayed-release membrane and/or the controlled-release component is a controlled-release membrane.

In one embodiment, the blocking agent is released continuously to provide therapeutic levels for at least four hours for a period between the hours of 12:00 midnight and 12:00 noon, preferably between 4:00 AM and 12:00 noon, more preferably between 5:00 AM and 12:00 noon, and most preferably between 6:00 AM and 11:00 A.M.

In one embodiment, the blocking agent administered comprises from about 50 to about 450 mg of propranolol or a salt thereof, preferably about 60 to about 320 of propranolol or a salt thereof, and more preferably about 80 to about 160 mg of propranolol or a salt thereof. In a most preferred embodiment, the blocking agent administered comprises about 80 mg of propranolol or a salt thereof or about 120 mg of propranolol or salt thereof.

These, and other embodiments of this invention are described in more detail in the detailed description of the invention that follows.

### DETAILED DESCRIPTION OF THE INVENTION

According to the present invention, it has been discovered that a β-adrenergic-blocking agent is effective for preventing and for treating morning migraine. The β-adrenergic-blocking agent can be administered prior to bedtime (e.g., between 8:00 PM and 12:00 midnight) to be effective in the early hours of each morning so that the biological availability of the effects of the blocking agent will last during the morning time period during which a person is prone to morning migraine. Preferably, the β-adrenergic-blocking agent is in a delayed and/or controlled release form. Thus, the blocking agent can be taken by a person before going to bed, and the blocking agent will not become biologically available until it is released over a period of the morning hours, such as for example from 12:00 midnight to 12:00 noon, preferably 4:00 AM to 12:00 noon, or any chosen periods of time there between, preferably from 6:00 AM to 11:00 AM.

In contrast to the prior art procedure involving constant prophylactic dosages of blocking agent, the present invention involves a targeted administration during a specific period in the day, in particular during the morning hours. The drug is administered such that it is effective during these hours. This permits a far more direct and less intrusive use of β-adrenergic-blocking agents. They may be administered in a reduced dosage amount as compared to a continuous prophylactic procedure. As a consequence, occurrences of adverse drug reaction, overdosage and/or discontinuance reaction are markedly reduced. This increases the availability of this form of therapy for many classes of individuals suffering from migraine. In addition, the incidents of fatigue are reduced according to the administration regime of the invention, as compared to other products and administration regimes, for example Inderal® LA.

In accordance with the present invention, the daily dosage requirements for β-adrenergic-blocking agents are reduced. In the prior art, it was customary to provide sufficient blocking agent to maintain an effective concentration in the blood stream over an extended time. Doses would be administered chronically, each day for weeks or even months. The present invention, on the other hand, requires an effective blood concentration for a shorter time period during each day. Administration is specific to a particular time period during the day during which morning migraine attacks are most likely to occur. Consequently, less blocking agent is required than is necessary for a continuous level of blocking agent through a day.

For example, for the targeted method of the present invention, the preferred dose is normally from about 60 to about 320 mg of propranolol hydrochloride per day, preferably about 80 to about 160 mg of propranolol hydrochloride per day, and most preferably about 80 or about 120 mg of propranolol hydrochloride per day.

The preferred administration of the β-adrenergic-blocking agent is once per day, in the evening (such as between 6:00 PM and 12:00 midnight), preferably before bedtime (such as between 8:00 PM and 12:00 midnight, preferably about 10:00 PM).

It is important to insure the entry of the β-adrenergic-blocking agent into the bloodstream at the appropriate time of day, namely during morning hours.

Minor amounts of other ingredients such as tonicity agents (e.g. NaCl), pH adjusters (e.g., a base such as NaOH, acids such as citric), emulsifiers or dispersing agents, buffering agents, preservatives, wetting agents, thickening agents (e.g. polyvinyl alcohol) and gelling agents (e.g. polaxamer) may also be present. Particularly preferred compositions contain sufficient amounts of the foregoing and/or other ingredients to be a substantially isotonic and/or buffered to a physiologically acceptable pH.

As previously discussed, the efficacy of a β-adrenergic-blocking agent is dependent upon its presence at the desired site of drug activity. This is commonly reflected by its concentration in the blood of the subject being treated. It is therefore particularly significant that the present administration of β-adrenergic-blocking agent is characterized by a pronounced blood concentration during morning hours, as compared to conventional procedures of oral administration. With the present invention, Cₘₐₓ is advantageously achieved between the hours of 8:00 am and 2:00 pm, preferably between 9:00 am and 12:00 noon.

To maximize its efficacy, it is desirable that the presence of β-adrenergic-blocking agent in a therapeutically effective amount be maintained over a substantial period of time. Thus it is preferred to sustain an effective blood concentration of blocking agent for at least two morning hours, preferably at least three morning hours, more preferably at least four, five, six, seven or more morning hours. This may be ensured by using a delayed and/or controlled release formulation.

Any effective controlled and/or delayed release enhancing compounds can be utilized in the formulation. Also, the delayed release mechanism and/or components are preferably in the form of a coating but can take the form of any other effective vehicle. While the controlled release mechanism and/or components are preferably in the form or a coating or a matrix, but can also be in the form of an any other effective vehicle or procedure, such as extrusion/spheronization of a mixture comprising the blocking agent and pharmaceutically acceptable excipients, as known in the art. See, e.g., J. W. Conine et al., Drug & Cosmetic Ind. 106, 38-41 (1970), hereby incorporated by reference.

The controlled and delayed release formulations can be made of two or three (or more) parts. The first part is a central core which can contain the blocking agent or can be coated with a coating (the second part in the three part formulation) that contains the blocking agent, for example in association with conventional excipients. The second part (or third part in the three part formulation) can be a coating, for example a polymeric coating which envelops or substantially envelops the central core. This coating is responsible for giving the blocking agent its particular controlled and/or delayed release characteristics. The central core (or the second part of the three part formulation) may be prepared by a number of techniques known in the art. Typically the blocking agent is bound to an inert carrier with a conventional binding agent. The inert carrier is typically a starch or sugar sphere. Sugar spheres are preferred but any pharmaceutically acceptable inert carrier may be utilized.

The binding agent that is used to secure the blocking agent can be any of the known binding agents. Examples of suitable lubricants that can be used include white wax, castor oil, palmitic acid, stearic acid, mineral oil, polyethylene glycol, etc. Examples of suitable coating agents that can be used include ethyl cellulose, methylcellulose, carboxymethylcellulose, hydroxypropymethylcellulose, polyvinylpyrrolidone, polymerized acrylates, etc. Other conventional pharmaceutical excipients may be incorporated into the binding agent.

The second (or third) component is the coating, for example a polymeric coating. The coating is responsible for giving the blocking agent its particular release characteristics. The coating may be produced, for example, from polymerized acrylates or copolymers of acrylic acid and methacrylic acid or esters of either monomer (hereinafter polymerized acrylates). The polymeric coating of the delayed release pellet may also be prepared from one of the organosiloxane oral coating materials known in the art such as polydimethylsiloxane, polydiethylsiloxane, etc.

Polymerized acrylates as well as copolymers of acrylic acid and methacrylic acid or esters of either monomer are known in the art and are available from many commercial sources. Examples of such copolymers include poly(methyl methacrylate), poly(ethyl methacrylate), poly(butyl methacrylate), poly(isobutyl methacrylate), poly(isobutyl methacrylate), poly(phenyl methacrylate) etc. The polymeric coating may optionally contain a sufficient quantity of a suitable plasticizer. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate.

The polymeric coating may also be made from a variety of coating materials that are typically utilized in the pharmaceutical arts. The coating may be manufactured from a variety of water insoluble polymers such as, for example, ethylcellulose, cellulose acetate, cellulose propionate, cellulose acetate butyrate, polyethylene, polypropylene, polyethylene oxide, polyvinyl acetate, polyvinyl chloride, etc. A minor proportion of a water soluble polymer may also be included in the polymeric coating. Examples of such polymers include methyl cellulose, hydroxypropyl cellulose, polyethylene glycol, polyvinyl pyrrolidone, etc. These coatings may also include conventional excipients such as plasticizers, antifoaming agents, antiadherants, etc.

The polymeric coating may be applied to the central core using methods and techniques known in the art. Typically a suspension, emulsion, or solution of the polymeric coating is prepared as is known in the art. The amount of fluidized polymeric coating required in the coating process may be readily calculated depending upon the amount of polymeric coating desired. The fluid polymeric coating may be applied to the central core by a number of coating techniques known in the art. Examples of suitable coating devices include fluid bed coaters, pan coaters, etc.

The sustained-release forms of administration according to the invention can also contain the blocking agent in a sustained-release matrix, preferably as a uniform distribution.

Matrix materials which can be used are physiologically compatible, hydrophilic materials known to those skilled in the art. The hydrophilic matrix materials used are preferably polymers and particularly preferably cellulose ethers, cellulose esters and/or acrylic resins. The matrix materials used are very particularly preferably ethyl cellulose, hydroxypropyl methyl cellulose, hydroxypropyl cellulose, hydroxymethyl cellulose, poly(meth)acrylic acid and/or derivatives thereof such as their salts, amides or esters.

Other preferred matrix materials are those consisting of hydrophobic materials such as hydrophobic polymers, waxes, fats, long-chain fatty acids, fatty alcohols or corresponding esters or ethers, or mixtures thereof. The hydrophobic materials used are particularly preferably C₁₂-C₃₀ fatty acid mono- or diglycerides and/or C₁₂-C₃₀ fatty alcohols and/or waxes, or mixtures thereof.

It is also possible to use mixtures of the above-mentioned hydrophilic and hydrophobic materials as the sustained-release matrix material.

The sustained-release matrix can be prepared by the conventional methods known to those skilled in the art.

Those skilled in the art will be aware that a therapeutically effective amount of a particular β-adrenergic-blocking agent will vary with the particular drug as well as the type, age, size, weight and general physical condition of the subject. The amount will also vary dependent upon the particular therapeutic effect desired. Typically, however, the present dosage can be less than that currently employed for analogous continuous prophylactic treatment.

Any of the β-adrenergic-blocking agents known in the art may be utilized in accordance with the present invention. This includes blocking agents in their basic states or as their acid addition salts. Certain β-adrenergic-blocking agents are, however, preferred. These include propranolol, nadolol, timolol, metoprolol, atenolol, labetolol, pindolol, oxprenolol and their salts. Of these, propranolol (or a salt thereof), particularly propranolol hydrochloride, is most preferred.

The most preferred embodiments of this invention utilize, as the blocking agent, the product InnoPran XL™ produced by Reliant Pharmaceuticals LLC. Innopran XL™ is in the form of capsules that contain 80 or 120 mg of active ingredient propranolol hydrochloride along with sugar spheres, ethylcellulose, povidone, hypromellose phthalate, diethyl phthalate, hypromellose, polyethylene glycol, gelatin, titanium dioxide and black iron oxide. The 120 mg capsules also contain yellow iron oxide.

Having thus described a presently preferred embodiment of the present invention, it will be appreciated that the objects of the invention have been achieved, and it will be understood by those skilled in the art that changes in construction and widely differing embodiments and applications of the invention will suggest themselves without departing from the spirit and scope of the present invention. The disclosure and description herein are intended to be illustrative and are not in any sense limiting of the invention.

We claim:

1. A method for treating a patient that suffers from morning migraines comprising administering a pharmaceutical composition containing a β-adrenergic-blocking agent and one or more pharmaceutically acceptable excipients to the patient in an amount sufficient to provide a therapeutically effective amount of said blocking agent in the morning to prevent or treat morning migraines.

2. The method of claim 1, wherein the blocking agent is propranolol, nadolol, timolol, metoprolol, atenolol, labetolol, pindolol, oxprenolol or a salt thereof.

3. The method of claim 1, wherein the blocking agent is propranolol or a salt thereof.

4. The method of claim 3, wherein the blocking agent is propranolol hydrochloride.

5. The method of claim 1, wherein an effective amount of blocking agent is available for a time period between approximately 12:00 midnight to 12:00 noon.

6. The method of claim 5, wherein an effective amount of blocking agent is available for a time period between approximately 4:00 AM to 12:00 noon.

7. The method of claim 6, wherein an effective amount of blocking agent is available for a time period between approximately 6:00 AM to 11:00 AM.

8. The method of claim 5, wherein the time period is at least four hours.

9. The method of claim 1, wherein an effective amount of blocking agent is sustained for at least four morning hours.

10. The method of claim 1, wherein the blocking agent is administered in a delayed and/or controlled release form.

11. The method of claim 1, wherein the composition is administered once daily.

12. The method of claim 11, wherein the composition is administered in the evening.

13. The method of claim 12, wherein the composition is administered between 8:00 PM and midnight.

14. The method of claim 13, wherein the composition is administered at about 10:00 PM.

15. The method of claim 1, wherein the composition is administered orally.

16. The method of claim 15, wherein the composition is administered in the form of a pill or capsule.

17. The method of claim 16, wherein the pill or capsule contains a delayed-release component and a controlled-release component.

18. The method of claim 17 wherein the delayed-release component is a delayed-release membrane.

19. The method of claim 17, wherein the controlled-release component is a controlled-release membrane.

20. The method of claim 1, wherein the blocking agent is released continuously for at least four hours for a period between midnight and noon.

21. The method of claim 20, wherein the blocking agent is released continuously for at least four hours for a period between 4:00 AM and noon.

22. The method of claim 21, wherein the blocking agent is released continuously for at least four hours for a period between 6:00 AM and 11:00 AM.
